(19) United States
(12) Patent Application Publication
(10) Pub. No.: US 2023/0126987 A1
(43) Pub. Date: Apr. 27, 2023

(54) METHODS FOR TREATING CYTOKINE STORM SYNDROME AND RELATED DISEASES

(71) Applicant: Generos Biopharma Ltd., George Town (KY)

(72) Inventors: Xin-Yuan FU, Zhejiang (CN); Xinyu LIU, Zhejiang (CN); Yi ZHOU, Zhejiang (CN); Chengchen LUFEI, Zhejiang (CN)

(73) Assignee: Generos Biopharma Ltd., George Town (KY)

(21) Appl. No.: 17/914,288

(22) PCT Filed: Mar. 26, 2021
(86) PCT No.: PCT/CN2021/083345

§ 371 Date: Sep. 23, 2022

Related U.S. Application Data
(60) Provisional application No. 63/001,077, filed on Mar. 27, 2020.

(51) Int. Cl. A61K 31/454; A61K 31/366; A61P 31/04; A61P 37/06; A61P 31/14; A61K 45/06

(52) U.S. Cl. CPC A61K 31/454; A61K 31/366; A61K 45/06; A61P 31/04; A61P 31/14; A61P 37/06

(57) ABSTRACT

This disclosure provides methods of treating cytokine storm syndrome and related diseases, including infectious diseases such as COVID-19. In particular, this disclosure provides a method of treating cytokine storm syndrome (CSS) in a subject in need thereof comprising administering a therapeutically effective amount of a compound selected from the group consisting of pimozide, artemisinin and its derivatives, or a pharmaceutically acceptable salt, or a solvate thereof, to a subject in need thereof.

### CROSS-REFERENCE TO RELATED APPLICATIONS

This application claims the priority benefits of U.S. Provisional Application 63/001,077 filed Mar. 27, 2020, the entire contents of which are incorporated herein by reference.

### BACKGROUND

The term “cytokine storm” describes the aberrant production of soluble mediators and the accompanying immunopathology following severe viral and bacterial infections. Aberrant immune responses and cytokine production are associated with the pathogenesis of multiple disease states, which range from viral infection to neurological disorders. Despite a link of cytokine and chemokine levels with morbidity and mortality following these viral and bacterial infections, there are no effective therapeutic treatments have been developed to treat the pathology associated with cytokine storm.

The invention described herein addresses this need and provides methods of treating cytokine storm syndrome (CSS) and related diseases, including infectious diseases such as COVID-19.

### SUMMARY

Provided in one aspect is a method of treating cytokine storm syndrome (CSS) in a subject in need thereof comprising administering a therapeutically effective amount of a compound selected from the group consisting of pimozide and artemisinin and its derivatives, or a pharmaceutically acceptable salt, or a solvate thereof, to a subject in need thereof.

In some embodiments, the cytokine storm syndrome (CSS) is associated with cytokine release syndrome (CRS), familial hemophagocytic lymphohistiocytosis (FHLH), Epstein-Barr virus associated HLH (EBV-HLH), or systemic juvenile idiopathic arthritis associated macrophage activation syndrome (systemic JIA-MAS).

In some embodiments, the cytokine storm syndrome (CSS) is associated with sepsis or a related bacterial induced inflammation.

In some embodiments, the cytokine storm syndrome (CSS) is associated with an infectious disease selected from: (a) coronavirus disease 2019 (COVID-19); (b) severe acute respiratory syndrome (SARS); (c) Middle East respiratory syndrome (MERS); (d) influenza; (e) human immunodeficiency virus (HIV); (f) malaria; (g) tuberculosis; (h) dengue fever; (i) Ebola virus disease (EVD); (j) Hepatitis A, B, or C virus; (k) Nipah virus (NiV) infection; (1) plague; (m) pneumonia; (n) rabies; (o) Staphylococcal infection; (p) typhus fever; (q) Zika virus (ZIKV); (r) West Nile fever; (s) Vibrio parahaemolyticus enteritis; (t) various types of encephalitis; (u) tetanus; (v) listeriosis; (w) Lyme disease; (x) measles; (y) meningitis; (z) mumps; and (aa) pelvic inflammatory disease.

In some embodiments, the cytokine storm syndrome (CSS) is associated with a cell therapy selected from chimeric antigen receptor (CAR) T-cell or NK-cell therapy, or associated with an antibody therapy. In some embodiments, the cytokine storm syndrome (CSS) is associated with a gene therapy involving a viral delivery system.

In some embodiments, the compound is pimozide, or a pharmaceutically acceptable salt, or a solvate thereof.

In some embodiments, the compound is artemisinin or a derivative thereof, or a pharmaceutically acceptable salt, or a solvate thereof.

In some embodiments, the method further comprises administering a therapeutically effective amount of an antibody against IL-1α, IL-Iβ, IL-2, TNFα, IFNy, IL-6, GMCSF, M-CSF, IL-12 IL-17, IL-23, IL-28, type I IFNs, CCL2, CXCL8, CXCL9, CXCL10, CXCL11, CCL11, and their respective receptors.

In some embodiments, the method further comprises further comprising administering a therapeutically effective amount of an antibody against CD20, CD47, BLyS, APRIL, and their respective receptors.

In some embodiments, the method further comprises administering a therapeutically effective amount of a compound selected from chloroquine, hydroxychloroquine, remdesivir, favipiravir, lopinavir, ritonavir, fingolimod, darunavir, cobicistat, thalidomide, lenalidomide, tetrandrine, and methylprednisolone.

In some embodiments, the method further comprises administering a therapeutically effective amount of a Bruton’s tyrosine kinase (BTK) inhibitor. In some embodiments, the BTK inhibitor is selected from ibrutinib, zanubrutinib, and acalabrutinib.

In some embodiments, the method further comprises administering a therapeutically effective amount of a NF-kB inhibitor. In some embodiments, the NF-kB inhibitor is selected from TPCA-1, BOT-64, BMS 345541, SC-514, IMD-0354, BAY 11-7082, JSH-23, GYY4137, CV-3988, LY294002, wortmannin, and mesalamine.

Provided in one aspect is a method of treating cytokine storm syndrome (CSS) associated with COVID-19 in a subject in need thereof comprising administering a therapeutically effective amount of pimozide, or a pharmaceutically acceptable salt, or a solvate thereof, to a subject in need thereof.

In some embodiments, pimozide is administered to the subject in an amount between about 1 mg/kg of body weight to about 20 mg/kg of body weight per day. In some embodiments, pimozide is administered to the subject in an amount between about 1 mg/kg of body weight to about 5 mg/kg of body weight per day. In some embodiments, pimozide is administered to the subject in an amount between about 5 mg/kg of body weight to about 10 mg/kg of body weight per day. In some embodiments, pimozide is administered to the subject in an amount between about 0.1 mg/kg of body weight to about 20 mg/kg of body weight per day. In some embodiments, pimozide is administered to the subject in an amount between about 0.1 mg/kg of body weight to about 0.5 mg/kg of body weight per day. In some embodiments, pimozide is administered to the subject in an amount between about 0.1 mg/kg of body weight to about 0.3 mg/kg of body weight per day. In some embodiments, pimozide is administered to the subject in an amount of no more than about 0.3 mg/kg of body weight per day.

In some embodiments, the method further comprises administering a therapeutically effective amount of an antibody against IL-1α, IL-1β, IL-2, TNFα, IFNγ, IL-6, GMCSF, M-CSF, IL-12 IL-17, IL-23, IL-28, type I IFNs, CCL2, CXCL8, CXCL9, CXCL10, CXCL11, CCL11, and their respective receptors.

In some embodiments, the method further comprises administering a therapeutically effective amount of an antibody against CD20, CD47, BLyS, APRIL, and their respective receptors.

In some embodiments, the method further comprises administering a therapeutically effective amount of a compound selected from chloroquine, hydroxychloroquine, remdesivir, favipiravir, lopinavir, ritonavir, fingolimod, darunavir, cobicistat, thalidomide, lenalidomide, tetrandrine, and methylprednisolone.

In some embodiments, the method further comprises administering a therapeutically effective amount of a Bruton’s tyrosine kinase (BTK) inhibitor. In some embodiments, the BTK inhibitor is selected from ibrutinib, zanubrutinib, and acalabrutinib.

In some embodiments, the method further comprises administering a therapeutically effective amount of a NF-kB inhibitor. In some embodiments, the NF-kB inhibitor is selected from TPCA-1, BOT-64, BMS 345541, SC-514, IMD-0354, BAY 11-7082, JSH-23, GYY4137, CV-3988, LY294002, wortmannin, and mesalamine.

### BRIEF DESCRIPTION OF THE DRAWINGS

FIG. 1 shows the results of the immunoblotting analysis with the antibodies as indicated, where Jurkat cells were pretreated with various concentrations of pimozide (Nib1) Nib1 for 1 hour before stimulation with interferon beta (50 ng/ml) for 30 min. Equal amount of lysate were subject to immunoblotting analysis with antibodies as indicated.

FIGS. 2A and 2B show the cytokine levels of TNFα and IL-6, respectively, in serum as measured by an ELISA kit. Briefly, the Balb/c mice were pre-treated with pimozide (Nib1) and dexamethasone acetate (DEX) or control (DMSO) for 1 hour, followed by LPS stimulation (10 mg/kg, i.p.). Whole blood was collected at 4 hours after LPS injection, and the plasma was extracted for ELISA measurement. *indicates P<0.05, **indicates P<0.01, *** indicates P<0.001, and **** indicates P<0.0001.

FIG. 3 shows the human PBMC cytokine profiling in response to LPS following whether the human PBMC was treated with either vehicle (DMSO) or pimozide (Nib1). Human PBMC was isolated from healthy donors by standard procedures and cultured in RPMI1640 medium with 10% FBS at 37° C. for 12 h. Then the cells were incubated with DMSO vehicle or Nib1 (10 µM) for 1 h before being challenged with 100 ng/mL LPS for 4 h at 37° C. The supernatant was collected and subjected to Luminex multiplex beads based cytokine profiling assay. The data is presented to show the percentage of each cytokine level to DMSO treated samples (n=2, error bar means SD).

FIGS. 4A, 4B, and 4C show the cytokine levels of IL-1β, IL-6, and TNFα, respectively, in the whole blood cells as measured by RT-qPCR. The Balb/c mice were challenged with low dose of LPS (10 µg/mouse) via intraperitoneal injection for 4 days and treated with the oral dosing of control (DMSO; n=7), dexamethasone acetate (DEX; 3 mg/kg, n=7), or pimozide (Nib1; 0.6 mg/kg, n=7). At the end of experiment, whole blood cells were collected and the total RNA was extracted for RT-qPCR measurement of the cytokine mRNA. *indicates P<0.05, **indicates P<0.01, *** indicates P<0.001.

FIGS. 5A and 5B show the cytokine levels of IL-6 and TNFα, respectively, in the bronchoalveolar lavage fluid (BALF) were measured by ELISA kit. Briefly, the C57BL/6 mice (male, 6-week old) were challenged with airway LPS administration and treated with pimozide (Nib1), dexamethasone acetate (DEX), or DMSO for 4 days. Bronchoalveolar lavage fluids were collected at the end point for ELISA measurement of the indicated cytokines. (* indicates p<0.05; ** indicates p<0.01.).

### DETAILED DESCRIPTION

This disclosure is directed to method of treating cytokine storm syndrome and related diseases. Such diseases include cytokine release syndrome (CRS), sepsis, coronavirus disease 2019 (COVID-19), severe acute respiratory syndrome (SARS), influenza, human immunodeficiency virus (HIV), a disease associated cell therapy selected from chimeric antigen receptor (CAR) T-cell or NK-cell therapy, and a disease associated with a gene therapy involving a viral delivery system. The methods comprise administering a compound selected from pimozide, artemisinin, and related compounds thereof. Without being bound by any theory, such compounds are believed to suppress the induction of the pro-inflammatory cytokines in response to a bacterial or viral infection. In some instances, the compounds disclosed herein also produce a synergistic therapeutic effect when combined with any one of the antibodies or compounds disclosed herein.

### Cytokine Storm Syndromes

Cytokine storm syndromes (CSS) are a group of inflammatory disorders characterized by the final common result of overwhelming systemic inflammation, hemodynamic instability, multiple organ dysfunction, and potentially death. The hemophagocytic syndromes hemophagocytic lymphohistiocytosis (HLH) and macrophage activation syndrome (MAS) are two clinically similar CSS with an unknown degree of pathoetiology overlap. Among rheumatic diseases, systemic juvenile idiopathic arthritis (JIA) and its adult analog, adult-onset Still’s disease (AOSD), have the highest association with cytokine storm. Cytokine storm is also often called MAS as a reference to the activated macrophages often seen on tissue biopsy despite lack of evidence that these cells cause the syndrome while they may produce inflammatory cytokines in some cases. (See Schulert, G.S. and Grom, A. A., Pathogenesis of macrophage activation syndrome and potential for cytokine-directed, therapies. Annu Rev Med, 2015, 66:145-159.)

Before germ theory, the term “sepsis” was used to characterize all states of uncontrolled inflammation. Today “sepsis” refers to overwhelming inflammation in the context of a systemic infection. The term “cytokine storm syndrome” (CSS) was developed to accommodate the observation that multiple inflammatory causes can result in a disease that appears very similar to sepsis. The unifying feature of CSS is the clinical and laboratory phenotype suggestive of massive inflammation progressing to multiple organ dysfunction syndrome (MODS) and eventually death, a final common pathway.

The clinical constituents of this pathway may include fever, tachycardia, tachypnea, hypotension, malaise, generalized swelling, altered mental status, diffuse lymphadenopathy, organomegaly (particularly of the liver and spleen), and often erythematous or purpuric rash. To standardize hemodynamic management of CSS, criteria for Systemic Inflammatory Response Syndrome (SIRS) were proposed in 1992.

An analysis of the underlying pathoetiology of all CSS suggests that cytokine storm results from excessive proinflammatory stimuli and/or inadequate regulation of inflammation. Proinflammatory stimuli can include antigens, superantigens (compounds that trigger nonspecific but massive activation of T cell receptors), adjuvants such as toll-like receptor (TLR) ligands, allergens (antigens triggering an allergic response), and proinflammatory cytokines themselves. Anti-inflammatory mechanisms can be humoral or cellular and seek to dampen or terminate a proinflammatory pathway.

The types of syndromes associated with cytokine storm syndromes are outlined in the following table.

TABLE 1

| | | |
|---|---|---|
| Syndrome | Pathologic Effectors | Potential Precision Therapy |
| FHLH | CD8+ T cells, IFNγ, IL-33 | T cell inhibitors, IFNy neutralization, IL-33 receptor blockade |
| EBV-HLH | Viremia, IFNy | B cell-depleting therapy |
| Systemic JIA-MAS | IL-Iβ, myeloid cell autoinflammation, IFNγ | IL-1β blockade, IFNy neutralization |
| NLRC4-MAS | IL-18, IL-18-induced IFNγ | IL-Iβ binding protein, IFNy neutralization |
| Non-systemic JIA-MAS | Unknown | Unknown |
| CRS | IL-6, macrophages | IL-6 receptor blockade, IL-6 neutralization |
| Sepsis | Heterogeneous and multifactorial | More precise genotyping and phenotyping required |

FHLH = familial hemophagocytic lymphohistiocytosis; IFNγ = interferon-γ; IL-33 = interleukin-33; EBV-HLH = Epstein-Barr virus-associated HLH; systemic JIA-MAS = systemic juvenile idiopathic arthritis-associated macrophage activation syndrome; CRS = cytokine release syndrome.

Cytokines are a diverse group of small proteins secreted by the cells for intercellular signaling and communication. Specific cytokines have autocrine, paracrine, and/or endocrine activity and, can elicit a variety of responses through receptor binding depending upon the cytokine and the target cell. Cytokines also control cell proliferation and differentiation and regulate angiogenesis and immune and inflammatory responses. The major types and actions of cytokines are outlined in the following table.

TABLE 2

| | |
|---|---|
| Type | Actions |
| Interferons | Regulation of innate immunity, activation of antiviral properties, antiproliferative effects |
| Interleukins | Growth and differentiation of leukocytes; many are proinflammatory |
| Chemokines | Control of chemotaxis, leukocyte recruitment; many are proinflammatory |
| Colony-stimulating factors | Stimulation of hematopoietic progenitor cell proliferation and differentiation |
| Tumor necrosis factor | Proinflammatory, activates cytotoxic T lymphocytes |

### CSS and Acute Lung Injury

Inflammation associated with a cytokine storm begins at a local site and spreads throughout the body via the systemic circulation. Signs of acute inflammation include rubor (redness), tumor (swelling or edema), calor (heat), dolor (pain), and “functio laesa” (loss of function). When localized in skin or other tissue, these responses increase blood flow, enable vascular leukocytes and plasma proteins to reach extravascular sites of injury, increase local temperatures (which is advantageous for host defense against bacterial infections), and generate pain, thereby warning the host of the local responses. However, these responses often occur to the detriment of local organ function, particularly when tissue edema causes a rise in extravascular pressures and a reduction in tissue perfusion. Shortly after inflammation begins, compensatory repair processes are employed, and in many cases the repair process completely restores tissue and organ function. When the inflammation is severe or the primary etiological agent triggering inflammation damages local tissue structures, the healing occurs with fibrosis, which can result in persistent organ dysfunction.

Most commonly associated with suspected or proven infections in the lungs or other organs, acute lung injury (ALI) is a common consequence of a cytokine storm in the lung alveolar environment and systemic circulation. In humans, ALI is characterized by an acute mononuclear/neutrophilic inflammatory response followed by a chronic fibroproliferative phase marked by progressive collagen deposition in the lung. Pathogen-induced lung injury can progress into ALI or its more severe form, acute respiratory distress syndrome (ARDS), as seen with SARS-CoV and influenza virus infections. See Huang, K.J. et al., An interferon-gamma-related cytokine storm in SARSpatients, J. Med. Virol., 2005, 75(2): 185-194. IL-1β is a key cytokine driving proinflammatory activity in bronchoalveolar lavage fluid of patients with lung injury.

The cytokine storm is best exemplified by severe lung infections, in which local inflammation spills over into the systemic circulation, producing systemic sepsis, as defined by persistent hypotension, hyper- or hypothermia, leukocytosis or leukopenia, and often thrombocytopenia. Viral, bacterial, and fungal pulmonary infections all cause the sepsis syndrome, and these etiological agents are difficult to differentiate on clinical grounds. In some cases, persistent tissue damage without severe microbial infection in the lungs also is associated with a cytokine storm and the clinical manifestations mimic sepsis syndrome. In addition to lung infections, the cytokine storm is a consequence of severe infections in the gastrointestinal tract, urinary tract, central nervous system, skin, joint spaces, and other sites. See Tisoncik, J.R. et al., Into the eye of the cytokine storm. Microbiol. Mol. Biol. Rev., 2012, 76(1):16-32.

Studies of patients with severe sepsis due to pulmonary or nonpulmonary infections show characteristic plasma cytokine profiles, which change over time. The acute-response cytokines TNF and IL-1β and the chemotactic cytokines IL-8 and MCP-1 appear in the early minutes to hours after infection, followed by a more sustained increase in IL-6. The anti-inflammatory cytokine IL-10 appears somewhat later, as the body attempts to control the acute systemic inflammatory response. IL-6 concentrations in peripheral blood have been used to assess the intensity of systemic cytokine responses in patients with sepsis, because IL-6 production is stimulated by TNF and IL-1β, providing an integrated signal of these two early-response cytokines. In the current COVID-19 epidemic, cytokine storm syndrome was detected in the patients and IL-6R blocking therapy has been used in severe case of SARS-COV-2 infection. See Huang, C. et al., Lancet, 2020, 395(10223):497-506.

### Current Treatments and Challenges

Infectious diseases remain a very real threat, accounting for approximately half of all deaths across the world. Malaria, tuberculosis, HIV disease, influenza, dengue, and emerging infections all contribute to morbidity and mortality. Characterized by a powerful and potentially destructive immune response, acute infections may be treated by targeting this immune response in order to reduce the self-inflicted damage initiated by the host in response to infection. Yet to date, successful targeting of the immune system during acute infections has proven to be unsuccessful and difficult.

The following table summarizes the immunomodulatory drugs that diminish inflammation during infection with drug treatment show therapeutic benefit. See Tisoncik, J.R. et al., Into the eye of the cytokine storm. Microbiol. Mol. Biol. Rev., 2012, 76(1):16-32.

TABLE 3

| | | |
|---|---|---|
| Type of therapeutic | Drug(s) | Immunomodulatory effect(s) |
| COX inhibitors | Mesalamine, celecoxib | Co-administration of COX inhibitors with zanamivir diminished cellular infiltrate and improved survival of H5N1 virus-infected mice compared to antiviral treatment alone |
| CCR2 inhibitor | PF-04178903 | Increased survival of mice infected with influenza virus and reduced lung immunopathology |
| Sphingosine receptor agonists | | Suppresses cytokine and chemokine production; sphingosine receptors have been shown to play an important role in innate immune responses |
| Anti-TNF agents | | Mediator of pulmonary inflammation during influenza A viral pneumonia; decreased severity of pulmonary immunopathology and prolonged survival of A/PR/8-infected mice |
| Statins | Simvastatin | Statins were not found to reduce the risk of developing severe disease in patients with pandemic influenza (H1N1) 2009 |
| OX40 | OX40-Ig fusion proteins | OX40 plays a critical role in T-cell-mediated immunopathology in the lung during viral infection; ligation on activated T cells reduces pulmonary eosinophilia during Cryptococcus neoformans infection |
| PPARα/PPARγ agonists | Gemfibrozil, pioglitazone, rosiglitazone, 15d-PGJ2, ciglitazone, troglitazone | 15d-PGJ2, ciglitazone, and troglitazone decreased production of IL-1α, IL-6, and TNF cytokines, CXCL8 and CCL5 chemokines, and ICAM-1 in RSV-infected lung epithelial cells; administration of gemfibrozil (intraperitoneally) on days 4 to 10 after exposure to H2N2 influenza virus and following the onset of illness significantly increased survival in mice with severe influenza |

As with most inflammatory diseases, cytokine storm can be treated effectively with corticosteroids. Methylprednisolone, which is used in most rheumatic diseases, has been the most widely reported in treatment of MAS, whether associated with systemic JIA or with SLE. In contrast, dexamethasone is often used in the treatment of FHLH and is the recommended agent for FHLH treatment. However, the use of corticosteroids often requires extended periods of high-dose steroid treatment and is complicated by an equally broad range of adverse effects.

In addition to the conventional anti-inflammation therapies, blockade of key cytokines like TNFα, IL-1β, IL-6, IFNγ have been tested in clinics. However, multiple large scale clinical trials on TNFα, IL-1β neutralizing antibodies have not been successful indicating the complexity of CSS. Also ablation of cell population responsible for cytokine storm is also emerging, such as T cell, B cell ablation therapy. JAK/STAT signaling is a common mechanism used by many different cytokine receptors, including IFNγ. Studies by two different groups showed efficacy of JAK inhibition in murine models of FHLH and MAS. Behrens, E.M. and G.A. Koretzky, Review: Cytokine Storm Syndrome: Looking Toward the Precision Medicine Era. Arthritis Rheumatol, 2017. 69(6):1135-1143.

### Therapeutic Compounds

This disclosure provides methods of treating CSS and related diseases using one or more therapeutic compounds.

In some embodiments, the methods disclosed herein include the use of pimozide and/or artemisinin. Also included are the derivatives, semi-synthetic derivatives, pharmaceutically acceptable salts, solvates, prodrugs, or stereisomers of these compounds.

Pimozide is a cell-permeable and orally available diphenylbutylpiperidine class of psychotropic drug with antagonistic activity against DAT (dopamine transporter) as well as several postsynaptic receptors, including D₂, D₃, D₄, and 5-HT₇ receptors, and works by blocking the action of dopamine. Pimozide is a FDA-approved for treating uncontrolled movements (motor tics) or outbursts of words/sounds (vocal tics) in patients with Tourette syndrome when other medicines have not worked. The chemical name for pimozide is 1-[1-[4,4-bis(4-fluorophenyl)butyl]-4-piperidinyl]-1,3-dihydro-2H-benzimidazole-2-one with the molecular formula of C₂₈H₂₉F₂N₃O and molecular weight of 461.56. Pimozide is associated with CAS No. 2062-78-4 and has the following structure:

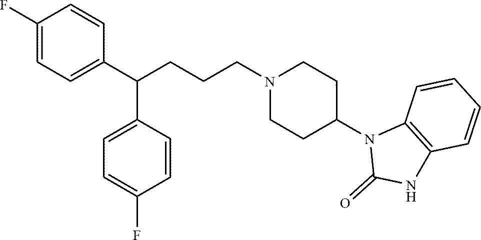 [SMILES: O=c1[nH]c2ccccc2n1C1CCN(CCCC(c2ccc(F)cc2)c2ccc(F)cc2)CC1]

Artemisinin and its derivatives (semi-synthetic derivatives) are compounds isolated from the plant Artemisia annua, sweet wormwood, a herb employed in Chinese traditional medicine and are used for treating malaria caused by Plasmodium falciparum. Artemisinin is a sesquiterpene lactone containing an unusual peroxide bridge (endoperoxide 1,2,4-trioxane ring), which is responsible for the drug’s mechanism of action. Artemisinin is associated with CAS No. 63968-64-9 and has a chemical formula of C₁₅H₂₂O₅ and molecular weight of 282.33. The chemical structure of artemisinin is:

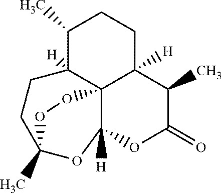 [SMILES: C[C@@H]1CC[C@H]2[C@@H](C)C(=O)O[C@@H]3O[C@]4(C)CC[C@@H]1[C@]32OO4]

Compounds that are related to artemisinin include, but are not limited to, dihydroartemisinin (DHA), artemether, artesunate, artemisone, arteether, and artelinic acid.

The present inventors found that in some instances, pimozide at a low dose provided similar or better anti-inflammatory efficacy than an anti-inflammatory corticosteroid, such as dexamethasone. Specifically, as described in the examples, pimozide significantly reduced the levels of lipopolysaccharide (LPS) induced pro-inflammatory cytokines, such as IL-6, IL-1β, GMCSF, IL17A, IL4 and IL23. In some embodiments, administration of any one of the therapeutic compounds described herein significantly reduces the levels of one or more of the following cytokines: IL-6, IL-1β, GMCSF, IL17A, IL4 and IL23. In some embodiments, administration of any one of the therapeutic compounds described herein, such as pimozide, significantly reduces the levels of IL-6 and IL-1β.

### Combination Therapy

In the methods described herein, in addition to the therapeutic compound described in the foregoing section (such as pimozide), a therapeutically effective amount of a suitable antibody may be co-administered. Antibodies against any interferons, interleukins, chemokines, colony-stimulating factors, and tumor necrosis factors associated with CSS are contemplated for use. Suitable antibodies include, but are not limited to, antibodies against IL-1α, IL-1β, IL-2, TNFα, IFNγ, IL-6, granulocyte-macrophage colony-stimulating factor (GMCSF), macrophage colony-stimulating factor (M-CSF), IL-12 IL-17, IL-23, IL-28, type I IFNs, CCL2, CXCL8, CXCL9, CXCL10, CXCL1 1, CCL11, and their respective receptors and/or ligands. Additional antibodies include, but are not limited to antibodies against CD20, CD47, B lymphocyte stimulator (BLyS), a proliferation-inducing ligand (APRIL), and their respective receptors and/or ligands.

In the methods described herein, in addition to the therapeutic compound described in the foregoing section (such as pimozide), a therapeutically effective amount of another compound may be co-administered. Suitable examples of additional compounds include, but are not limited to, chloroquine, hydroxychloroquine, remdesivir, favipiravir, lopinavir, ritonavir, fingolimod, darunavir, cobicistat, thalidomide, lenalidomide, tetrandrine, and methylprednisolone.

Other additional compounds to be co-administered include a Bruton’s tyrosine kinase (BTK) inhibitor or a nuclear factor kappa B (NF-kB) inhibitor. Examples of BTK inhibitors include, but are not limited to, ibrutinib, zanubrutinib, and acalabrutinib. Examples of NF-kB inhibitors, include but are not limited to, TPCA-1 (5-(4-fluorophenyl)-2-ureidothiophene-3-carboxamide, CAS No. 507475-17-4); BOT-64 (6,6-dimethyl-2-(phenylimino)-6,7-dihydro-5H-benzo-[1,3]oxathiol-4-one, CAS No. 113760-29-5); BMS 345541 (N-(1,8-dimethylimidazo[1,2-a]quinoxalin-4-yl)-1,2-ethanediamine hydrochloride, CAS No. 547757-23-3); SC-514 (4-amino-[2,3″]bithiophenyl-5-carboxylic acid amide, CAS No. 354812-17-2); IMD-0354 (N-(3,5-bis-trifluoromethylphenyl)-5-chloro-2-hydroxybenzamide, CAS No. 978-62-1); BAY 11-7082 ((E)-3-(4-methylphenyl)sulfonylprop-2-enenitrile, CAS No. 19542-67-7); JSH-23 (4-methyl-N1-(3-phenylpropyl)-1,2-benzenediamine, CAS No. 749886-87-1); GYY4137 ((p-methoxyphenyl)morpholino-phosphinodithioic acid; CAS No. 106-140-09-4); CV-3988 (rac-3-(N-Octadecylcarbamoyl)-2-Methoxy) propyl-(2-thiazolioethyl) phosphate, CAS No. 85703-73-7); LY294002 (2-(4-morpholino)-8-phenyl-4H-1-benzopyran-4-one, CAS No. 154447-36-6); wortmannin; and mesalamine.

### Methods of Treatment

Provided in one aspect is a method of treating cytokine storm syndrome (CSS) in a subject in need thereof comprising administering a therapeutically effective amount of a compound selected from the group consisting of pimozide and artemisinin and its derivatives, or a pharmaceutically acceptable salt, or a solvate thereof, to a subject in need thereof.

In some embodiments, the cytokine storm syndrome (CSS) is associated with cytokine release syndrome (CRS). In some embodiments, the cytokine storm syndrome (CSS) is associated with sepsis. In some embodiments, the cytokine storm syndrome (CSS) is associated with sepsis or a related bacterial induced inflammation. In some embodiments, such inflammation is caused by a bacteria induced disease. In some embodiments, the cytokine storm syndrome (CSS) is associated with familial hemophagocytic lymphohistiocytosis (FHLH). In some embodiments, the cytokine storm syndrome (CSS) is associated with Epstein-Barr virus associated HLH (EBV-HLH). In some embodiments, the cytokine storm syndrome (CSS) is associated with systemic juvenile idiopathic arthritis associated macrophage activation syndrome (systemic JIA-MAS).

In some embodiments, the cytokine storm syndrome (CSS) is associated with an infectious disease. Examples of infectious diseases include, but are not limited to, coronavirus disease 2019 (COVID-19); severe acute respiratory syndrome (SARS); Middle East respiratory syndrome (MERS); influenza; human immunodeficiency virus (HIV); malaria; tuberculosis; dengue fever; Ebola virus disease (EVD); Hepatitis A, B, or C virus; Nipah virus (NiV) infection; plague; pneumonia; rabies; Staphylococcal infection; typhus fever; Zika virus (ZIKV); West Nile fever; Vibrio parahaemolyticus enteritis; various types of encephalitis; tetanus; listeriosis; Lyme disease; measles; meningitis; mumps; and pelvic inflammatory disease. In some embodiments, the cytokine storm syndrome (CSS) is associated with coronavirus disease 2019 (COVID-19).

In some embodiments, the cytokine storm syndrome (CSS) is associated with a cell therapy selected from chimeric antigen receptor (CAR) T-cell or NK-cell therapy, or associated with an antibody therapy. In some embodiments, the cytokine storm syndrome (CSS) is associated with a gene therapy involving a viral delivery system.

In some embodiments, the compound is pimozide, or a pharmaceutically acceptable salt, or a solvate thereof.

In some embodiments, the compound is artemisinin or a derivative thereof, or a pharmaceutically acceptable salt, or a solvate thereof. Examples of such derivatives include, but are not limited to, dihydroartemisinin (DHA), artemether, artesunate, artemisone, arteether, and artelinic acid.

In some embodiments, the method further comprises administering a therapeutically effective amount of an antibody against IL-1α, IL-1β, IL-2, TNFα, IFNy, IL-6, GMCSF, M-CSF, IL-12 IL-17, IL-23, IL-28, type I IFNs, CCL2, CXCL8, CXCL9, CXCL10, CXCL11, CCL11, and their respective receptors.

In some embodiments, the method further comprises further comprising administering a therapeutically effective amount of an antibody against CD20, CD47, BLyS, APRIL, and their respective receptors.

In some embodiments, the method further comprises administering a therapeutically effective amount of a compound selected from chloroquine, hydroxychloroquine, remdesivir, favipiravir, lopinavir, ritonavir, fingolimod, darunavir, cobicistat, thalidomide, lenalidomide, tetrandrine, and methylprednisolone.

In some embodiments, the method further comprises administering a therapeutically effective amount of a Bruton’s tyrosine kinase (BTK) inhibitor. In some embodiments, the BTK inhibitor is selected from ibrutinib, zanubrutinib, and acalabrutinib.

In some embodiments, the method further comprises administering a therapeutically effective amount of a NF-kB inhibitor. In some embodiments, the NF-kB inhibitor is selected from TPCA-1, BOT-64, BMS 345541, SC-514, IMD-0354, BAY 11-7082, JSH-23, GYY4137, CV-3988, LY294002, wortmannin, and mesalamine.

Provided in one aspect is a method of treating cytokine storm syndrome (CSS) associated with COVID-19 in a subject in need thereof comprising administering a therapeutically effective amount of pimozide, or a pharmaceutically acceptable salt, or a solvate thereof, to a subject in need thereof.

In some embodiments, pimozide is administered to the subject in an amount between about 1 mg/kg of body weight to about 20 mg/kg of body weight per day. In some embodiments, pimozide is administered to the subject in an amount between about 0.03 mg/kg of body weight to about 10 mg/kg of body weight per day. In some embodiments, pimozide is administered to the subject in an amount between about 0.03 mg/kg of body weight to about 0.3 mg/kg of body weight per day. In some embodiments, pimozide is administered to the subject in an amount between about 0.3 mg/kg of body weight to about 1 mg/kg of body weight per day. In some embodiments, pimozide is administered to the subject in an amount between about 1 mg/kg of body weight to about 5 mg/kg of body weight per day. In some embodiments, pimozide is administered to the subject in an amount between about 5 mg/kg of body weight to about 10 mg/kg of body weight per day. In some embodiments, pimozide is administered to the subject in an amount between about 0.1 mg/kg of body weight to about 0.5 mg/kg of body weight per day. In some embodiments, pimozide is administered to the subject in an amount between about 0.5 mg/kg of body weight to about 1 mg/kg of body weight per day. In some embodiments, pimozide is administered to the subject in an amount between about 0.1 mg/kg of body weight to about 0.3 mg/kg of body weight per day. In some embodiments, pimozide is administered to the subject in an amount of no more than about 0.3 mg/kg of body weight per day, or no more than about 0.5 mg/kg of body weight per day, or no more than about 0.7 mg/kg of body weight per day, or no more than about 1 mg/kg of body weight per day.

In some embodiments, the method further comprises administering a therapeutically effective amount of an antibody against IL-1α, IL-1β, IL-2, TNFα, IFNy, IL-6, GMCSF, M-CSF, IL-12 IL-17, IL-23, IL-28, type I IFNs, CCL2, CXCL8, CXCL9, CXCL10, CXCL11, CCL11, and their respective receptors.

In some embodiments, the method further comprises administering a therapeutically effective amount of an antibody against CD20, CD47, BLyS, APRIL, and their respective receptors.

In some embodiments, the method further comprises administering a therapeutically effective amount of a compound selected from chloroquine, hydroxychloroquine, remdesivir, favipiravir, lopinavir, ritonavir, fingolimod, darunavir, cobicistat, thalidomide, lenalidomide, tetrandrine, and methylprednisolone.

In some embodiments, the method further comprises administering a therapeutically effective amount of a Bruton’s tyrosine kinase (BTK) inhibitor. In some embodiments, the BTK inhibitor is selected from ibrutinib, zanubrutinib, and acalabrutinib.

In some embodiments, the method further comprises administering a therapeutically effective amount of a NF-kB inhibitor. In some embodiments, the NF-kB inhibitor is selected from TPCA-1, BOT-64, BMS 345541, SC-514, IMD-0354, BAY 11-7082, JSH-23, GYY4137, CV-3988, LY294002, wortmannin, and mesalamine.

### Dosing/Administration

Any one of the active agents disclosed herein, such as pimozide, may be administered to the subject in an amount between about 0.1 mg/kg of body weight to about 20 mg/kg of body weight. In some embodiments, the active agent is administered to the subject in an amount between about 0.1 mg/kg of body weight to about 10 mg/kg of body weight per day. In some embodiments, the active agent is administered to the subject in an amount between about 0.1 mg/kg of body weight to about 1 mg/kg of body weight per day. In some embodiments, the active agent is administered to the subject in an amount between about 0.1 mg/kg of body weight to about 0.5 mg/kg of body weight per day. In some embodiments, the active agent is administered to the subject in an amount between about 0.1 mg/kg of body weight to about 0.3 mg/kg of body weight per day. In some embodiments, the active agent is administered to the subject in an amount of no more than about 0.3 mg/kg of body weight per day. In some embodiments, the active agent is administered to the subject in an amount between about 1 mg/kg of body weight to about 5 mg/kg of body weight per day. In some embodiments, the active agent is administered to the subject in an amount between about 1 mg/kg of body weight to about 20 mg/kg of body weight per day. In some embodiments, the active agent is administered to the subject in an amount between about 1 mg/kg of body weight to about 5 mg/kg of body weight per day. In some embodiments, the active agent is administered to the subject in an amount between about 5 mg/kg of body weight to about 10 mg/kg of body weight per day.

The compounds of the present disclosure may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. It will be appreciated that the route used may vary with, for example, the condition of the recipient. Where the compound is administered orally, it may be formulated as a pill, capsule, tablet, etc. with a pharmaceutically acceptable carrier or excipient. Where the compound is administered parenterally, it may be formulated with a pharmaceutically acceptable parenteral vehicle and in a unit dosage injectable form.

### Pharmaceutical Compositions

Also, the compounds disclosed herein may also be formulated as a pharmaceutical composition comprising a compound of the present disclosure in association with a pharmaceutically acceptable diluent or carrier. The carrier, diluent or excipient used will depend upon the means and purpose for which the compound of the present disclosure is being applied.

The pharmaceutical compositions of the invention are formulated, dosed and administered in a fashion, i.e., amounts, concentrations, schedules, course, vehicles and route of administration, consistent with good medical practice. Factors for consideration in this context include the disorder being treated, the mammal being treated, the clinical condition of the individual patient, the cause of the disorder, the site of delivery of the agent, the method of administration, the scheduling of administration, and other factors known to medical practitioners. The therapeutically effective amount of the compound to be administered will be governed by such considerations, and is the minimum amount necessary to ameliorate or treat the disorder. The compound of the present disclosure can be formulated into pharmaceutical dosage forms to provide an easily controllable dosage of the drug and to enable patient compliance with the prescribed regimen.

Pharmaceutical formulations of the compounds of the present disclosure may be prepared for various routes and types of administration. For example, a compound of the present disclosure having the desired degree of purity may optionally be mixed with pharmaceutically acceptable diluents, carriers, excipients or stabilizers in the form of a lyophilized formulation, a milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The formulations may be prepared using conventional dissolution and mixing procedures. For example, the bulk drug substance (i.e., a compound of the present disclosure or stabilized form of the compound) can be dissolved in a suitable solvent in the presence of one or more excipients.

Solvents can be generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride, benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, argirune, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations may also include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament).

### Definitions

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The term “acceptable” with respect to a formulation, composition or ingredient, as used herein, means having no persistent detrimental effect on the general health of the subject being treated.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different times.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutically acceptable” indicates that the substance or composition is compatible chemically and/or toxicologically with the other ingredients comprising a formulation, and/or the mammal being treated therewith.

The phrase “pharmaceutically acceptable salt,” unless otherwise indicated, includes salts that retain the biological effectiveness of the corresponding free acid or base of the specified compound and are not biologically or otherwise undesirable. Examples of pharmaceutically acceptable salts include those salts prepared by reaction of the compounds of the present disclosure with a mineral or organic acid or an inorganic base, such salts including, but not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyn-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, g -hydroxybutyrates, glycollates, tartrates, methanesulfonates, propanesulfonates, naphthalene- 1-sulfonates, naphthalene-2-sulfonates, citrates, and mandelates. Since a single compound of the present disclosure may include more than one acidic or basic moiety, the compounds of the present disclosure may include mono, di or tri-salts in a single compound.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, solvates refer to compounds that contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol.

A “stereoisomer” refers to a compound made up of the same atoms bonded by the same bonds but having different three-dimensional structures, which are not interchangeable. It is therefore contemplated that various stereoisomers and mixtures thereof and includes “enantiomers,” which refers to two stereoisomers whose molecules are nonsuperimposeable mirror images of one another.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The present invention, thus generally described, will be understood more readily by reference to the following examples, which are provided by way of illustration and are not intended to be limiting of the present invention.

### EXAMPLES

### Example 1. STAT5 Inhibition In Human T Cell Line Jurkat Cells

STAT5 is a key transcription factor in T cell development, including the newly discovered Th-GM lineage cells. Th-GM is characterized by the IL-7 induced STAT5 activation in CD4+ T cells and subsequently upregulated expression of GM-CSF and IL-3. They have been shown to be the critical subset of T helper cells to promote the autoimmunity including multiple sclerosis. (See Sheng, W., et al., STAT5 programs a distinct subset of GM-CSF-producing T helper cells that is essential for autoimmune neuroinflammation. Cell Res, 2014. 24(12):p. 1387-402.) The genetic ablation of Stat5 genes in CD4 T cells has rendered the animal more resistant to adjuvant-induced arthritis progression. (See WO2016048247). In response to virus infection, interferons are readily induced through activation of innate immunity and turn on a plethora of anti-virus genes through the activation of JAK-STAT pathways including STAT5.

The objective of this study was to evaluate the in vitro effect of pimozide (Nib1) on the activation of STAT5 in the human T cell line Jurkat cells.

Pimozide was purchased from Sigma (P1793, 98% purity). DMSO was used as the control vehicle. Jurkat cells (ATCC, TIB152) were maintained in RPMI medium supplemented with 10% fetal bovine serum (FBS) and 1% antibiotics in humidified incubator supplied with 5% CO₂ at 1×10^6/ml density. The cells were incubated at 37° C. for 12 hours before stimulation. Then pimozide (Sigma, P1793) was added at various concentrations 1 hour before cells were stimulated with 50 ng/ml human IFNβ (Sino Biological, 10704-HNAS) for 30 min. Cells were harvested by centrifugation at 300 g for 5 min, washed with PBS, and lysed in RIPA lysis buffer. An equal amount of cell lysate was resolved on SDS-PAGE and subjected to immunoblotting analysis with anti-STAT5 total and pY694 antibodies (Cell Signaling Technology, 9351#) and GAPDH (Sangon Biotech, B661104) antibody.

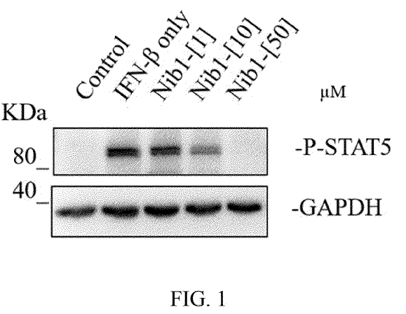

FIG. 1 shows the results of the immunoblotting analysis with the antibodies as indicated, where Jurkat cells were pretreated with various concentrations of pimozide (Nib1) for 1 hour before stimulation with interferon beta (50 ng/ml) for 30 min.

The STAT5 activation in Jurkat cells was observed as indicated by the phosphorylation at the critical tyrosine residue in SH2 domain when IFNβ was added to cell medium. The dose response of pimozide was shown with various concentrations used. When T cells were pretreated with 10 µM pimozide for 1 hour, the activation of STAT5 was reduced by more than 50%. Phospho-STAT5 signal was completely undetectable when cells were treated with 50 µM pimozide.

The results of this study show that pimozide functioned as STAT5 inhibitor in human T cell culture system in vitro. IFNβ induced STAT5 activation is an important signaling even in response to virus infection. Pimozide dramatically inhibited STAT5 phosphorylation which is the marker of its activation.

### Example 2. Attenuation of the Secretion of Pro-Inflammatory Cytokines in Lipopolysaccharide Induced Sepsis Model in Mice With Pimozide

Sepsis is the life threating disorder through uncontrolled response to infection including bacteria and viruses. (See, Schulte, W., J. Bernhagen, and R. Bucala, Mediators Inflamm, 2013, 2013: p. 165974). Lipopolysaccharides (LPS) are the unique components of Gram negative bacteria and are recognized by Toll like receptor (TLR) 4 and CD14 complex on innate immunity cells like macrophages, monocytes, fibroblasts. (See, Poltorak, A., et al., Science, 1998, 282(5396): p. 2085-8; and Muta, T. and K. Takeshige, Eur J Biochem, 2001, 268(16): p. 4580-9.) LPS is widely used as the potent agent for septic shock model in rodents. The so-called “cytokine storm” is believed to play a critical role in the pathogenesis of sepsis and acute respiratory distress syndrome (ARDS) seen in coronavirus including SARS-Cov-2 virus infection. (See, Mehta, P., et al., Lancet, 2020. 395(10229): p. 1033-1034; Tisoncik, J.R., et al., Microbiol Mol Biol Rev, 2012. 76(1): p. 16-32; and Vaninov, N., Nat Rev Immunol, 2020. 20(5): p. 277.) Tumor necrosis factor α (TNFα) and interleukin 6 (IL 6) are among the major proinflammatory cytokines released in the process of cytokine storm which if unchecked will lead to cardiovascular collapse and multiple organ failure in patients with critical situation of sepsis and ARDS. (See, Leon, L.R., A.A. White, and M.J. Kluger, Am J Physiol, 1998. 275(1): p. R269-77; and Moore, J.B. and C.H. June, Science, 2020. 368(6490): p. 473 474.)

The objective of this study was to assess the anti-inflammatory effect of pimozide (Nib1), in the model of LPS-induced sepsis which resembles the massive response seen in cytokine storm of SARS-CoV-2 infection. Multiple cytokines are known to activate Janus kinase (JAK) and STAT pathways to execute their biological functions. (See O'Shea, J.J., M. Gadina, and R.D. Schreiber, Cell, 2002. 109 Suppl: p. S121 31.) This study demonstrates that pimozide (Nib1) alleviated the cytokine storm in general.

Pimozide (Nib1) was purchased from SIGMA (Cat.: P1793, Lot: SLBX0707); Dexamethasone acetate tablets (DEX) were purchased from Xianju Pharma (NMPN: H33020822). The control vehicle was dimethyl sulfoxide (DMSO).

Forty-two 6-8-week-old female Balb/c mice (SLAC Animal Technology Co. Ltd, Shanghai, China) were randomly divided into DMSO Vehicle (n=14), DEX (dexamethasone) group (n=14), and Nib1 (pimozide) group (n=14). Animals of each group were intraperitoneally (i.p.) injected with DMSO (equal volume as drug treatment), or dexamethaone (DEX, 3 mg/kg, 0.6 mg/mL), or pimozide (Nib1, 5 mg/kg, 1 mg/mL), respectively. After 1 hour, all the animals were exposed to lethal dose of LPS (10 mg/kg, 2 mg/mL, i.p., Solarbio L8880). All animals were sacrificed 4 hours after LPS injection for tissue and whole blood collection. Plasma was extracted following standard procedure and subject to ELISA detection of IL-6 and TNFα according to the manufacturer’s instruction (Absin, cat. # 520004-96T and 520010-96T).

To elucidate the inhibitory effect of pimozide (Nib1) on LPS-induced septic shock, mice were treated with Nib1 1 hour before intraperitoneal injection of LPS. The plasma cytokine levels of TNFα and IL-6 in the serum of mice at 4 hours after LPS injection were assayed. 10 mg/kg LPS stimulation dramatically elevated the levels of TNFα and IL-6 in the plasma, compared to unstimulated animals (undetectable, data not shown). Pretreatment with supratherapeutic dose of DEX almost abolished the secretion of TNFα. and IL-6 levels at 4 hours (p<0.0001, p<0.01 respectively), suggesting the experiment setting was successful as literature report. Pimozide (Nib1) pretreatment significantly reduced the plasma levels of TNFα and IL-6 (p<0.01, p<0.001 respectively).

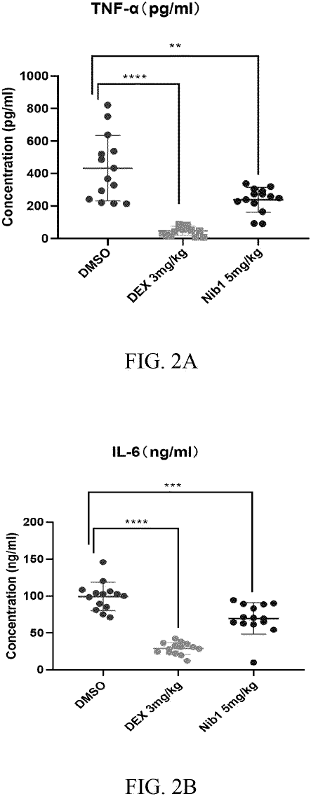

FIGS. 2A and 2B show the cytokine levels of TNFα and IL-6, respectively, in the serum as measured by an ELISA kit. Briefly, the Balb/c mice were pre-treated with pimozide (Nib1) and dexamethasone acetate (DEX) or control (DMSO) for 1 hour, followed by LPS stimulation (10 mg/kg, i.p.). Whole blood was collected at 4 hours after LPS injection and plasma was extracted for ELISA measurement. *indicates P<0.05, **indicates P<0.01, *** indicates P<0.001, and **** indicates P<0.0001.

This study successfully established the LPS-induced sepsis model in mice. Pimozide (Nib1) pre-treatment significantly suppressed the secretion of LPS-induced pro-inflammatory cytokines, such as IL-6 and TNFα. The variation between individual animals in DMSO and pimozide (Nib1) group were relatively large and is consistent with literature reports.

### Example 3. Attenuation of the Secretion of Pro-Inflammatory Cytokines in Lipopolysaccharide-Induced Cytokine Storm Model in Human Peripheral Blood Mononuclear Cells With Pimozide

The objective of this study was to assess the anti-inflammatory effect of pimozide (Nib1), in a model of LPS-induced cytokine release in human PBMC which resembles the massive response seen in cytokine storm of SARS-CoV-2 infection. This study demonstrates that pimozide (Nib1) alleviated the cytokine storm in general.

Pimozide (Nib1) was purchased from SIGMA (Cat.: P1793, Lot: SLBX0707). The control vehicle was dimethyl sulfoxide (DMSO).

hPBMCs were isolated by density gradient centrifugation on Ficoll-Paque PLUS (Solarbio, Cat. No.: P8900) from freshly collected EDTA blood. Cells from the interphase were harvested, washed, and cultured in 24-well plates at 1.25 × 10⁶ cells per well in RPMI 1640 medium (TransGen Biotech, Cat. No.: FI201-01), which was supplemented with 1% penicillin-streptomycin (TransGen Biotech, Lot# M40912) and 10% Fetal Bovine Serum (HyClone, Cat. No.: SV30160.03). The cultures were incubated overnight at 37° C. in a humidified atmosphere with 5% CO₂.

On the next day, pimozide (Nib1) (10 µM) was added to cells as prophylactic intervention. The cultures were incubated for 1 hour at 37° C. in a humidified atmosphere with 5% CO₂. hPBMC were then stimulated with LPS (100 ng/ml) and were incubated at 37° C. in a humidified atmosphere with 5% CO₂. The cultured cells and culture supernatants were harvested after 4 hours. The cytokines in supernatant were measured by Luminex multiplex beads based assay.

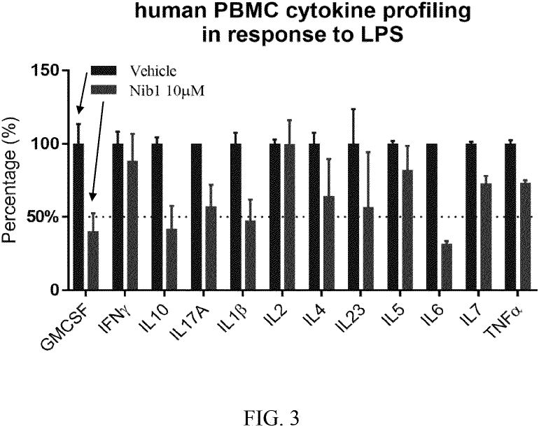

The results showed that the human PBMCs produced multiple cytokines in response to LPS stimulation at varied levels (raw data not shown) and the levels of each cytokine in DMSO-treated samples were normalized as 100% (See FIG. 3). Prophylactic dosing of pimozide (Nib1) significantly reduced the secretion of several key proinflammatory cytokines in response to LPS stimulation, including IL-6, IL-1β, GMCSF, IL17A, IL4 and IL23, as the relative levels of these cytokines dropped dramatically in the presence of pimozide (Nib1).

The results of this study clearly demonstrate that pimozide (Nib 1) is a potent anti-inflammatory agent and attenuated the production of multiple inflammatory cytokines in a well-established in vitro human cellular assay in the condition mimicking the septic shock induced by LPS.

### Example 4. Attenuation of the Secretion of Pro-Inflammatory Cytokines in Lipopolysaccharide-Induced Sepsis Model in Mice With Low Doses of Pimozide

The objective of this study was to assess the anti-inflammatory effect of pimozide (Nib1) in a model of repetitive LPS challenge caused sepsis-like persistent inflammation, which resembles the long-dragged inflammatory process seen in SARS-CoV-2 infection. Compared to the acute phase sepsis study described in Example 3, the objective for this study was to assess the efficacy of clinically safe dose of pimozide (Nib1) in five days, which is similar to the time frame of clinical intervention for COVID19 patients.

Pimozide (Nib1) was purchased from SIGMA (Cat.: P1793, Lot: SLBX0707). Dexamethasone acetate tablets (DEX) was purchased from Xianju Pharma (NMPN: H33020822). Dimethyl sulfoxide (DMSO) was used as the control vehicle. LPS was obtained from Solarbio (Cat.: L8880).

Balb/c mice (6-8-week-old female, ~20 g weight, SLAC Animal Technology Co. Ltd, Shanghai, China) were randomly divided into 3 groups and injected intraperitoneally with 10 µg LPS / mouse every day for four days. The animals were gavaged with control (DMSO; n=7), dexamethasone acetate (DEX; 3 mg/kg, n=7), or pimozide (Nib1; 0.6 mg/kg, n=7) daily for five days. At the end of the experiment, all animals were sacrificed, and whole blood cells were purified by centrifugation and subjected to standard RT-qPCR detection of IL-6 and TNFα mRNA levels according to the manufacturer’s instructions.

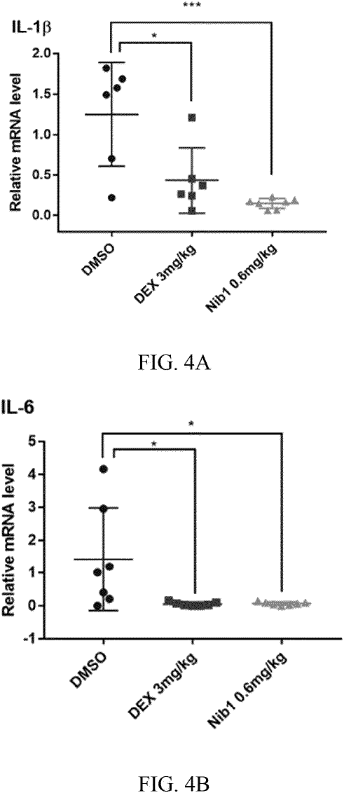

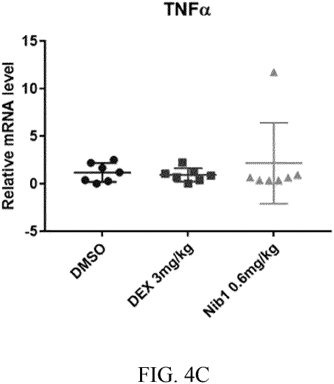

To elucidate the inhibitory effect of pimozide (Nib1) on LPS-induced chronic inflammation, mice were subjected to low dose of LPS via intraperitoneal injection for 4 days and treated with control (DMSO), dexamethasone acetate (DEX), or pimozide (Nib1) as indicated in FIGS. 4A, 4B, and 4C via oral administration. As shown in FIGS. 4A and 4B, supratherapeutic dose of DEX almost abolished the secretion of IL-1 β and IL-6 levels (p<0.05 both), suggesting the experiment setting was successful as literature reports. A low dose of pimozide (Nib1) treatment significantly reduced the plasma levels of IL-1 β and IL-6 (p<0.001, p<0.05 respectively), with better or similar effect compared to DEX. In this study, FIG. 4C shows that the TNFα level returned to basal level, suggesting it is a cytokine induced at early or acute phase of LPS challenge as shown in Example 3 and literature reports. Therefore, no difference was detected for different treatment groups.

This study evaluated the effect of low dose pimozide (Nib1) treatment on relatively mild but persistent inflammation caused by low dose systemic LPS exposure, which better recapitulates the clinical scenario in the COVID19 patients with mild to moderate symptoms. Surprisingly, the low dose of pimozide (Nib1) suppressed the secretion of LPS-induced pro-inflammatory cytokines, such as IL-1 β and IL-6. The anti-inflammation effect of pimozide (Nib1) was even better or at least similar to the powerful DEX treatment at relatively higher dosage. As such, these results demonstrate the therapeutic value of clinically acceptable dosages of pimozide (Nib1) in patients with systemic inflammation.

### Example 5. Attenuation of the Secretion of Pro-Inflammatory Cytokines in Lipopolysaccharide-Induced Acute Lung Injury Model With Low Doses of Pimozide

Acute lung injury (ALI) is a common consequence of a cytokine storm in the lung alveolar environment and systemic circulation and is most commonly associated with suspected or proven infections in the lungs or other organs. In humans, ALI is characterized by an acute mononuclear/neutrophilic inflammatory response followed by a chronic fibroproliferative phase marked by progressive collagen deposition in the lung. Pathogen-induced lung injury can progress into ALI or its more severe form, acute respiratory distress syndrome (ARDS), as seen with SARS-CoV and influenza virus infections. (See, Huang, K.J., et al., J Med Virol, 2005. 75(2): p. 185-94). Lipopolysaccharides (LPS) are the unique components of Gram-negative bacteria and are recognized by Toll-like receptor (TLR) 4 and CD14 complex on innate immunity cells like macrophages, monocytes, fibroblasts. (See, Poltorak, A., et al., Science, 1998, 282(5396): p. 2085-8; and Muta, T. and K. Takeshige, Eur J Biochem, 2001. 268(16): p. 4580-9.) LPS is widely used as the potent agent for acute lung injury model in rodents. Tumor necrosis factor (1 (TNFα) and interleukin 6 (IL-6) are among the major proinflammatory cytokines in bronchoalveolar lavage fluid of patients with lung injury. (See, Leon, L.R., A.A. White, and M.J. Kluger, Am J Physiol, 1998. 275(1): p. R269-77).

The objective of this study was to assess the anti-inflammatory effect of pimozide (Nib1), in the model of LPS induced acute lung injury, which resembles the massive response seen in cytokine storm of SARS-CoV-2 infection. Multiple cytokines are known to activate Janus kinase (JAK) and STAT pathways to execute their biological functions. (See O'Shea, J.J., M. Gadina, and R.D. Schreiber, Cell, 2002. 109 Suppl: p. S121 31.) This study demonstrates that pimozide (Nib1) alleviated the cytokine storm in general.

Pimozide (Nib1) was purchased from SIGMA (Cat.: P1793, Lot: SLBX0707). Dexamethasone acetate tablets (DEX) was purchased from Xianju Pharma (NMPN: H33020822). Dimethyl sulfoxide (DMSO) was used as the control vehicle. LPS was obtained from Solarbio (Cat.: L8880).

C57BL/6 male mice (8-week age, SLAC Animal Technology Co. Ltd, Shanghai, China) were randomly divided into DMSO Vehicle (po, qd, n=7), DEX (dexamethasone group; 3 mg/kg, po, qd, n=7) and pimozide (Nib1 group; 0.6 mg/kg, po,qd, n=7). Animals were sensitized with 1 µg LPS in 20 µl PBS into airway on day 1 only, and the test compounds or control were administered orally for 4 consecutive days. At the end of experiment, the mice were sacrificed and bronchoalveolar lavage fluids (BALF) were isolated following standard procedure. The levels of IL-6, TNFα in BALF were measured by ELISA according to the manufacturer’s instruction (Absin, cat. # 520004-96T and 520010-96T).

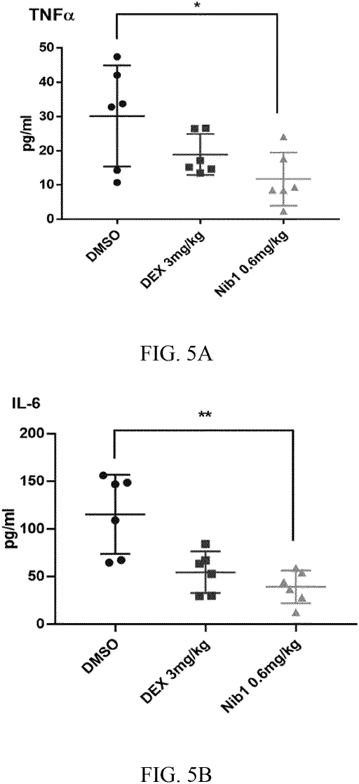

To elucidate the inhibitory effect of pimozide (Nib1) on LPS-induced acute lung injury model, mice were challenged with nasal administration of LPS and subjected to daily treatment with oral dosing of control vehicle, dexamethasone or pimozide for four days. This process resembled the clinical scenario of COVID19 patients who suffered the lung infection and were treated for a short period with anti-inflammation drugs. The cytokine levels of TNFα and IL-6 in the bronchoalveolar lavage fluids were measured by ELISA and are shown in FIGS. 5A and 5B, respectively. Compared to DMSO-treated group, 3 mg/kg DEX treatment reduced the secretion of TNFα and IL-6 levels by about 50% (p<0.05, p<0.05 respectively), which was consistent with literature reports. Surprisingly, low dose of Nib1 (0.6 mg/kg) treatment significantly reduced the levels ofTNFα and IL-6 (p<0.01, p<0.001 respectively). The effects on two cytokines by pimozide (Nib1) (0.6 mg/kg) intervention were slightly better than DEX treated group.

This study successfully established a LPS-induced acute lung injury model in mice. Clinically safe dose of pimozide (Nib1) treatment were shown to significantly suppress the secretion of LPS-induced pro-inflammatory cytokines, such as IL-6 and TNFα, in the lung aveolar chamber.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method of treating cytokine storm syndrome (CSS) in a subject in need thereof comprising administering a therapeutically effective amount of a compound selected from the group consisting of pimozide and artemisinin and its derivatives, or a pharmaceutically acceptable salt, or a solvate thereof, to a subject in need thereof.

2. The method of claim 1, wherein the cytokine storm syndrome (CSS) is associated with cytokine release syndrome (CRS), familial hemophagocytic lymphohistiocytosis (FHLH), Epstein-Barr virus associated HLH (EBV-HLH), or systemic juvenile idiopathic arthritis associated macrophage activation syndrome (systemic JIA-MAS).

3. The method of claim 1, wherein the cytokine storm syndrome (CSS) is associated with sepsis or a related bacterial induced inflammation.

4. The method of claim 1, wherein the cytokine storm syndrome (CSS) is associated with an infectious disease selected from:
(a) coronavirus disease 2019 (COVID-19);
(b) severe acute respiratory syndrome (SARS);
(c) Middle East respiratory syndrome (MERS);
(d) influenza;
(e) human immunodeficiency virus (HIV);
(f) malaria;
(g) tuberculosis;
(h) dengue fever;
(i) Ebola virus disease (EVD);
(j) Hepatitis A, B, or C virus;
(k) Nipah virus (NiV) infection;
(1) plague;
(m) pneumonia;
(n) rabies;
(o) Staphylococcal infection;
(p) typhus fever;
(q) Zika virus (ZIKV);
(r) West Nile fever;
(s) Vibrio parahaemolyticus enteritis;
(t) various types of encephalitis;
(u) tetanus;
(v) listeriosis;
(w) Lyme disease;
(x) measles;
(y) meningitis;
(z) mumps; and
(aa) pelvic inflammatory disease.

5. The method of claim 1, wherein the cytokine storm syndrome (CSS) is associated with a cell therapy selected from chimeric antigen receptor (CAR) T-cell or NK-cell therapy, or associated with an antibody therapy.

6. The method of claim 1, wherein the cytokine storm syndrome (CSS) is associated with a gene therapy involving a viral delivery system.

7. The method of claim 1, wherein the compound is pimozide, or a pharmaceutically acceptable salt, or a solvate thereof.

8. The method of claim 1, wherein the compound is artemisinin or a derivative thereof.

9. The method of claim 1, further comprising administering a therapeutically effective amount of an antibody against IL-1α, IL-1β, IL-2, TNFα, IFNγ, IL-6, GMCSF, M-CSF, IL-12 IL-17, IL-23, IL-28, type I IFNs, CCL2, CXCL8, CXCL9, CXCL10, CXCL11, CCL11, or a receptor thereof.

10. The method of claim 1, further comprising administering a therapeutically effective amount of an antibody against CD20, CD47, BLyS, APRIL, or a receptor thereof.

11. The method of claim 1, further comprising administering a therapeutically effective amount of a compound selected from the group consisting of chloroquine, hydroxychloroquine, remdesivir, favipiravir, lopinavir, ritonavir, fingolimod, darunavir, cobicistat, thalidomide, lenalidomide, tetrandrine, and methylprednisolone.

12. The method of claim 1, further comprising administering a therapeutically effective amount of a Bruton’s tyrosine kinase (BTK) inhibitor.

13. The method of claim 12, wherein the BTK inhibitor is selected from the group consisting of ibrutinib, zanubrutinib, and acalabrutinib.

14. The method of claim 1, further comprising administering a therapeutically effective amount of a NF-kB inhibitor.

15. The method of claim 14, wherein the NF-kB inhibitor is selected from the group consisting of TPCA-1, BOT-64, BMS 345541, SC-514, IMD-0354, BAY 11-7082, JSH-23, GYY4137, CV-3988, LY294002, wortmannin, and mesalamine.

16. A method of treating cytokine storm syndrome (CSS) associated with COVID-19 in a subject in need thereof comprising administering a therapeutically effective amount of pimozide, or a pharmaceutically acceptable salt, or a solvate thereof, to a subject in need thereof.

17. The method of claim 16, wherein pimozide is administered to the subject in an amount between about 0.1 mg/kg of body weight to about 0.5 mg/kg of body weight per day.

18. The method of claim 16, wherein pimozide is administered to the subject in an amount between about 0.1 mg/kg of body weight to about 0.3 mg/kg of body weight per day.

19. The method of claim 16, wherein pimozide is administered to the subject in an amount of no more than about 0.3 mg/kg of body weight per day.

20. The method of claim 16, wherein pimozide is administered to the subject orally or via parenteral injection.

21-28. (canceled)
